(19) United States
(12) Patent Application Publication
(10) Pub. No.: US 2010/0280476 A1
(43) Pub. Date: Nov. 4, 2010

(54) UNDERGARMENT LINERS

(76) Inventor: QUEENCY D. MATEO, SAN DIEGO, CA (US)

Correspondence Address: Jennifer J. Riel;Riel Law, 9550 S. Eastern Ave Suite 253, Las Vegas, NV, 89123, US

(21) Appl. No.: 12/772,153

(22) Filed: Apr. 30, 2010

Related U.S. Application Data
(60) Provisional application No. 61/174,503, filed on May 1, 2009.
(60) Provisional application No. 61/240,306, filed on Sep. 7, 2009.

(51) Int. Cl. A61F 13/56

(52) U.S. Cl. 604/385.05

(57) ABSTRACT

A disposable undergarment liner for preventing undergarment stains comprises (a) a nonwoven sheet that includes a top side, a bottom side, and an adhesive element permanently affixed to the bottom side and (b) a peel away paper that covers the adhesive element. The peel away paper is comprised of a glossy side removeably attached to the adhesive element and a matte side facing opposite the top side. The peel away paper is removed to expose the adhesive element and the adhesive element adheres onto the user's undergarment. The nonwoven sheet may be rectangular, triangular, or may further include a first portion, third portion, and second portion positioned between the first and third portions. The first portion is adapted for use within the user's posterior area, the second portion is adapted for use within the user's crotch area, and the third portion is adapted for use within the user's anterior area.

This application claims the benefit of priority under 35 U.S.C. §119 of provisional application Ser. No. 61/174,503 filed May 1, 2009 and provisional application Ser. No. 61/240,306 filed Sep. 7, 2009, the contents of which are hereby incorporated by reference.

### FIELD OF THE INVENTION

The present invention generally relates to hygiene products. More particularly, the present invention relates to undergarment liners that prevent stains on undergarments.

### BACKGROUND

Many people of various age ranges may experience bodily discharges, including light urinary leakage and fecal smears on clothing after defecation from restroom use. Such incidents do not warrant bulky and absorbable material as used in incontinence products or full-fledged diapers, which are inconvenient and noticeable. There are no adequate products that provide a thin, discreet protective barrier that is disposable and is designed to protect undergarments from stains and keep the undergarments clean.

Currently, none of the undergarment liners on the market specifically address stains from occurring, are developed to act as a disposable protective barrier for any article of clothing subject to urinary and/or fecal contact, and are thin enough to provide comfort while being undetectable by others. Those that are available for purchase are designed to primarily maximize fluid absorbency and are categorized as either incontinence products which are widely used by the geriatric population or as diapers used by infants and toddlers who have not yet developed bladder and bowel control.

None of the undergarment liners on the market are adapted for use by men of any age. Men of all ages and even teenagers are susceptible to chronic underwear staining even if they do not suffer from incontinence problems. The staining of men's underwear may be due to a variety of reasons such as: poor wiping techniques; poor hygiene; excess moisture due to humid climates; athletic and other physical activity causing excessive perspiration; digestive issues such as irritable bowel syndrome which cause frequent bowel movements; skin sensitivity, rashes, and other conditions making adequate wiping painful and impossible at times; or from the climbing of undergarments into the crevice of the user's buttocks. The products on the market are either designed for incontinence issues or for use by women with menstrual cycles. Even panty liners for women cannot be used for men to prevent stains since the dimensions, shapes and configurations of women's panty liners are not suitable for men, nor are they thin and discreet enough to prevent detection and discomfort.

Additionally, the undergarments liners currently on the market are not adapted for use by children. A child may be beyond the age of requiring full-fledged diapers (which absorb more but are consequently thicker, bulkier, more expensive, and noticeable), but may not yet be of the age to have full bladder or bowel control, or who have not yet mastered personal bathroom hygiene. There is often a transition period after children stop using diapers and start using the restroom on their own, where children do not often clean up or “wipe” adequately after urinating or defecating. There are no products that are designed to protect the child's undergarments during this transition period. Additionally, there are some children who may have difficulty controlling bladder and/or bowel functions due to feelings of anxiousness, fear, or excitement or due to not yet mastering complete bladder and/or bowel control.

Accordingly, there is a need in the art for a thin, discreet, and comfortable undergarment liner that is not meant to absorb large amounts of bodily fluids, but rather acts primarily to prevent stains from setting and merging onto any articles of fabric that the user may wear (for example, briefs, boxers, or panties). There is a need in the art for undergarment liners that are adapted for use by men and teenage boys to prevent staining of their undergarments. Furthermore, there is also a need in the art for an undergarment liner intended for use by children who stain their undergarments although they have outgrown diapers and know how to use restroom facilities. A product that addresses these needs in the art would allow the clothing or underwear to remain clean, retain its original color, and prevents the material from breaking down and disintegrating from the acidity from urine or fecal matter. Such a product would be designed to be thrown away after use, so the more expensive undergarments do not have to be.

### SUMMARY

The present invention provides a undergarment liner that is relatively flexible, and its thin, comfortable profile is designed to protect a user's clothing and undergarments from stains. The present invention is directed to an undergarment liner that is discreet, sleek and resilient and has a soft, cloth-like feel. It is difficult for a user to feel the difference between their underwear material and these undergarment liners which allows the user to feel as if they were not using any protection at all. Furthermore, the undergarment liner of the present invention is easy to apply and remove; and it allows a user to simply dispose of unwanted evidence or stained product. By preventing stains on underwear and pants, the present invention assists to prolong the life of clothing material. Getting rid of any type of stains may involve an extended amount of time and chemical detergents and manual friction on focal areas where the stain is concentrated. This product allows the user to bypass these steps, thus saving time on hand washing needs. Additionally, the present invention helps prevent cross contamination of soiled laundry by blocking the user's undergarments from absorbing fecal material and the likelihood of transferring feces onto other clothes that are simultaneously being washed.

This undergarment liner is absorbent, soft, and sterile on one side, and includes adhesive on the other side. It is applied to the inside of an undergarment, usually on the underside and rear of the undergarments, where fecal leakage or staining may occur. One embodiment of the present invention is directed to an undergarment liner adapted for use by men to prevent undergarment stains and is comprised of proper form, shape, and dimensions to be applied to the inside of a man's undergarment. Another embodiment of the present invention is directed to an undergarment liner adapted for use by children to prevent undergarment stains and is designed in proper form, shape, and dimensions to be applied to the inside of children's undergarments (e.g., briefs or boxers for boys or panties for girls). In both embodiments, the present invention may also extend or be positioned toward the front area of the user, where urinary leakage may occur as well. It is designed to protect the undergarment from stains resulting from small urinary or fecal discharge, or from inadequate cleanup or wiping after restroom use. Nonetheless, the present invention is designed to be relatively inexpensive and disposable. Additionally, the liner is designed to be discreet and non-bulky, so that observers would not notice that a man is wearing an underwear liner in embodiments adapted for use by men thereby avoiding embarrassment or that a child is wearing an undergarment liner under their clothing which is important for developing children to gain confidence, to feel like “big kids” and to avoid ridicule.

To achieve the foregoing and in accordance with the purposes of the present invention, the present invention is directed to a disposable undergarment liner for preventing undergarment stains and is comprised of (a) a nonwoven sheet that includes a top side, a bottom side, and an adhesive element permanently affixed to the bottom side; and (b) a peel away paper that covers the adhesive element wherein the peel away paper includes a glossy side removeably attached to the adhesive element and a matte side that faces opposite the top side. The undergarment liner of the present invention is secured to a user's undergarment by removing the peel away paper to expose the adhesive element and adhering the adhesive element directly onto the user's undergarment.

The nonwoven sheet may be comprised of a rectangular shape, a triangular shape, an oval shape, or any other shape suitable to adequately address the staining of undergarments. In other embodiments, the nonwoven sheet further comprises a first portion, a second portion, and a third portion wherein the second portion is positioned between the first portion and the third portion, and the second portion is narrower than the first portion and the third portion. The first portion is adapted to be placed in the posterior area of the user's underwear, the second portion is adapted to be placed in the crotch area of the user's underwear, and the third portion is adapted to be placed in the anterior area of the user's underwear. In some embodiments, the first portion and the third portion are the same shape and dimension thereby making the overall shape of the undergarment liner symmetrical and similar to an hour-glass-like shape which can simultaneously address both urinary and fecal stains in the anterior and posterior areas of the user's undergarment respectively. In further embodiments, the nonwoven sheet further comprises a second layer positioned below the top side of the nonwoven sheet and above the bottom side of the nonwoven sheet. This second layer is comprised of a material that is more dense than the nonwoven sheet and provides additional protection for user's with more severe staining issues. In some variations of the invention, the second layer may be comprised of a thin plastic sheet.

One embodiment of the present invention is directed the above-described disposable undergarment liner for use by men, and such embodiments are thus adapted for use by men and are sized, dimensioned, and shaped accordingly. The embodiments of the invention adapted for use by men mainly address posterior staining of undergarments although some embodiments may also address anterior staining.

Another embodiment of the present invention is directed the above-described disposable undergarment liner for use by children, and such embodiments are thus adapted for use by children and are sized, dimensioned, and shaped accordingly. The embodiments of the invention adapted for use by children can address posterior and/or posterior staining of undergarments. Some variations may address only posterior or anterior staining while other variations address both poster and anterior staining simultaneously. Additionally, other variations of the invention are versatile as such variations may be positioned within the undergarment liner for either posterior or anterior staining, or they may be positioned within the undergarment to address both posterior and anterior staining simultaneously.

The above description sets forth a summary of embodiments of the present invention so that the description that follows may be better understood and contributions of the present invention to the art may be better appreciated. Some of the embodiments of the present invention may not include all of the features or characteristics listed in the above summary. There may be, of course, other features of the invention that will be described below and may form the subject matter of claims. In this respect, before explaining at least one embodiment of the invention in further detail, it is to be understood that the invention is not limited in its application to the details of the construction and to the arrangement of the components set forth in the following description or as illustrated in the drawings. The invention is capable of other embodiments and of being practiced and carried out in various ways. Furthermore, it is to be understood that the phraseology and terminology employed herein are for the purpose of description and should not be regarded as limiting.

Other features, aspects, and advantages of the present invention will become apparent from the following description of the invention, taken in conjunction with the accompanying drawings, which illustrate, by way of example, various features of embodiments of the invention.

### BRIEF DESCRIPTION OF THE DRAWINGS

FIG. 1 depicts a top view of an undergarment liner in accordance with an embodiment of the present invention.

FIG. 2. depicts a perspective view from below the undergarment liner shown in FIG. 1 comprising of peel away paper to expose adhesive material in accordance with an embodiment of the present invention.

FIG. 3(a) depicts a rear view of the undergarment liner shown in FIG. 1 illustrating how the undergarment liner is secured onto the user's underwear in accordance with an embodiment of the present invention.

FIG. 3(b) depicts a side view of the undergarment liner shown in FIG. 1 illustrating how the undergarment liner is secured onto the user's underwear in accordance with an embodiment of the present invention.

FIG. 4 depicts a rear interior view of the user's pants and illustrates how the undergarment liner shown in FIG. 1 is secured onto the user's pants in accordance with an embodiment of the present invention.

FIG. 5 depicts a magnified transverse cross-sectional view of the undergarment liner shown in FIG. 1 in accordance with an embodiment of the present invention.

FIG. 6 depicts a top view of an undergarment liner in accordance with an embodiment of the present invention.

FIG. 7 depicts a top view of an undergarment liner in accordance with an embodiment of the present invention.

FIG. 8(a) depicts a top view of an undergarment liner in accordance with an embodiment of the present invention.

FIG. 8(b) depicts a rear view of underwear with the undergarment liner shown in FIG. 8(a) therein and illustrates where the undergarment liner is positioned onto the user's underwear in accordance with an embodiment of the present invention.

### DESCRIPTION OF THE INVENTION

In the following description of embodiments of the invention, reference is made to the accompanying drawings, which form a part of this application. The drawings show, by way of illustration, certain embodiments in which the invention may be practiced. It is to be understood that other embodiments may be utilized and modifications may be made without departing from the scope of the present invention. Also, it is to be understood that the phraseology and terminology employed herein are for the purpose of description and should not be regarded as limiting.

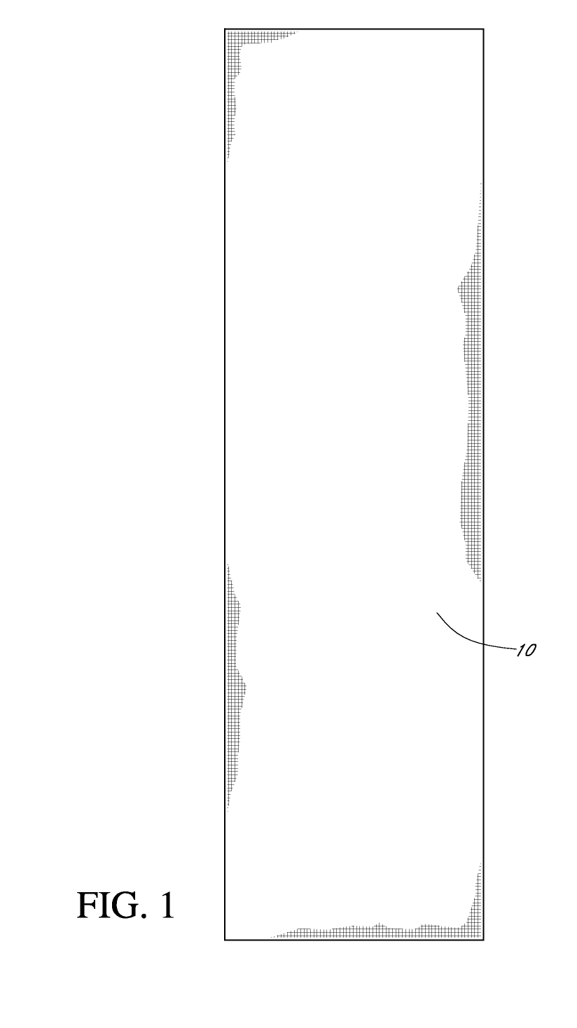

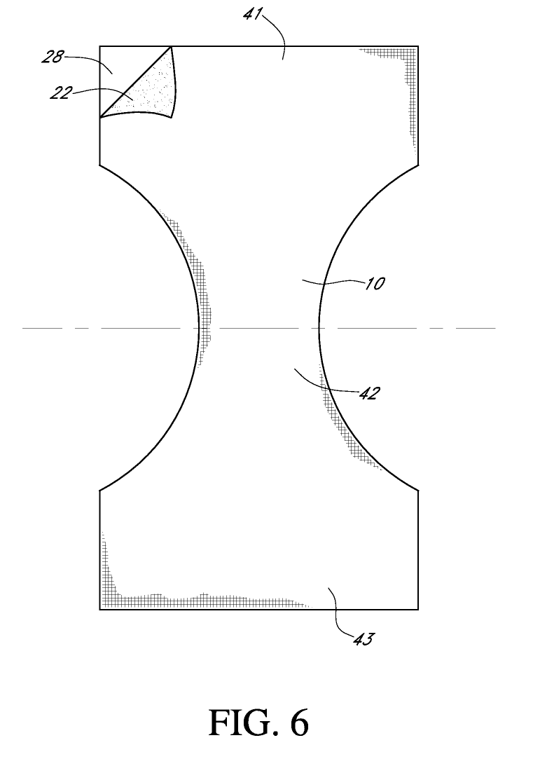

The embodiment of the present invention depicted in FIG. 1 is directed to an undergarment liner that generally is comprised of a sheet 10. In the embodiment shown in FIG. 1, sheet 10 is generally of a rectangular form although it may be comprised of a variety of shapes as exemplified in FIGS. 6-8(b). Sheet 10 is thin and in many embodiments of the present invention, sheet 10 is as thin as a sheet of paper or as thin as an adhesive bandage such as a BAND-AID®. Sheet 10 is made of a sterile, soft, and absorbent material. It is designed to protect the garment that it is covering from staining caused by mild urinary or fecal discharge.

In most embodiments of the invention, sheet 10 is made of a nonwoven fabric which is generally a sheet or web structures bonded together by entangling fiber or filaments mechanically, chemically, thermally or by solvent treatment. Since the present invention provides a liner that is disposable after each use, sheet 10 being comprised of nonwoven fabrics is ideal since such fabrics may be engineered and designed to have a limited life suitable as a single-use fabric. Additionally, nonwoven fabrics can provide specific functions such as absorbency, resilience, stretch, strength, and/or softness and are suitable as a bacterial barrier to provide sterility. Such properties may be combined to create an appropriate material for sheet 10 that is suited for stain prevention and as a barrier between the user and fecal or urine elements, while achieving a good balance between product use, life, and cost.

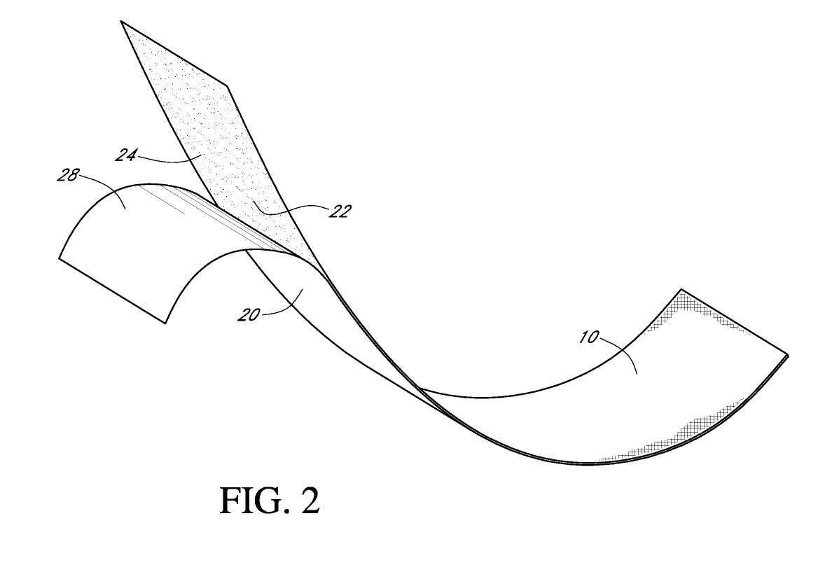

FIG. 2 depicts a perspective view from below the undergarment liner shown in FIG. 1. As shown in FIG. 2, the undergarment liner of the present invention is comprised of: sheet 10 that includes a bottom side 24, an adhesive element 22 that is permanently affixed to bottom side 24, and a peel away paper that covers adhesive element 22. The peel away paper includes a glossy side 28 and a matte side 20. Matte side 20 faces opposite the top side of sheet 10. Glossy side 28 is removeably attached to adhesive element 22 to allow the peel away paper to be removed from adhesive element 22 to expose adhesive element 22 for securing the undergarment liner to an undergarment liner. When a user wants to secure the undergarment liner to an undergarment, the user would simply remove the peel away to expose adhesive element 22 and the adhesive element 22 is then adhered directly onto the user's undergarment.

Adhesive element 22 may be comprised of any suitable adhesive known in the art such as hot melt glue, and it may be applied to the entire surface of bottom side 24 or to one or more portions of bottom side 24 in any pattern, configuration, and quantity that is sufficient to secure sheet 10 to the user's undergarment. For example, adhesive element 22 may be comprised of a series of adhesive lines and/or adhesive dots applied to bottom side 24, or adhesive element 22 may be comprised of a single line or strip of adhesive. Adhesive 22 is permanently affixed to bottom side 24 of sheet 10 so when a remover removes the undergarment liner from his undergarment, the adhesive is durable enough not to separate from bottom side 24 and does not remain on the undergarment. Adhesive 22's adhesive strength is strong enough to be permanently affixed to bottom side 24 and to securely attach sheet 10 to undergarments, but not too strong to allow the liner to be removable from the undergarment. The peel away paper is comprised of glossy side 28 and matte side 20; and FIG. 2 illustrates the peel away paper partially peeled from sheet 10 to expose glossy side 28 that is removeably attached to adhesive element 22 and matte side 20 that faces away from sheet 10. Once this peel away paper is removed and adhesive element 22 is exposed, sheet 10 may be attached to the inside of the user's undergarments as a liner to prevent stains.

In the embodiment shown in FIG. 2, the peel away paper is the same shape and dimension of sheet 10 although in other embodiments, the peel away paper may be a different shape or the same shape as sheet 10, and it may be larger or smaller than sheet 10. If the peel away paper is smaller than sheet 10, adhesive element 22 would be applied to only a portion of sheet 10, and the dimension and shape of the peel away paper would correspond with the dimension and shape of adhesive element 22 since the peel away paper functions to protect and maintain the stickiness of adhesive element 22 until sheet 10 is applied to a user's undergarment.

In an alternate embodiment, sheet 10 may further include a second layer that is positioned below the top side or surface of sheet 10 and above bottom side 24. The second layer would provide additional protection from stains and bodily fluids. The second layer may be made of a material that is more dense than the material of sheet 10 and may be made of a material that is closely woven. For instance, the second layer may be comprised of a thin plastic sheet or any other material that is dense enough to prevent stains and bodily fluids from leaking through and onto the undergarment if sheet 10 fails to do its job. Such embodiments that include a second layer would be suitable for users who suffer from more serious stain problems or for users who are experiencing temporary conditions such as frequent bowel movements caused by food poisoning.

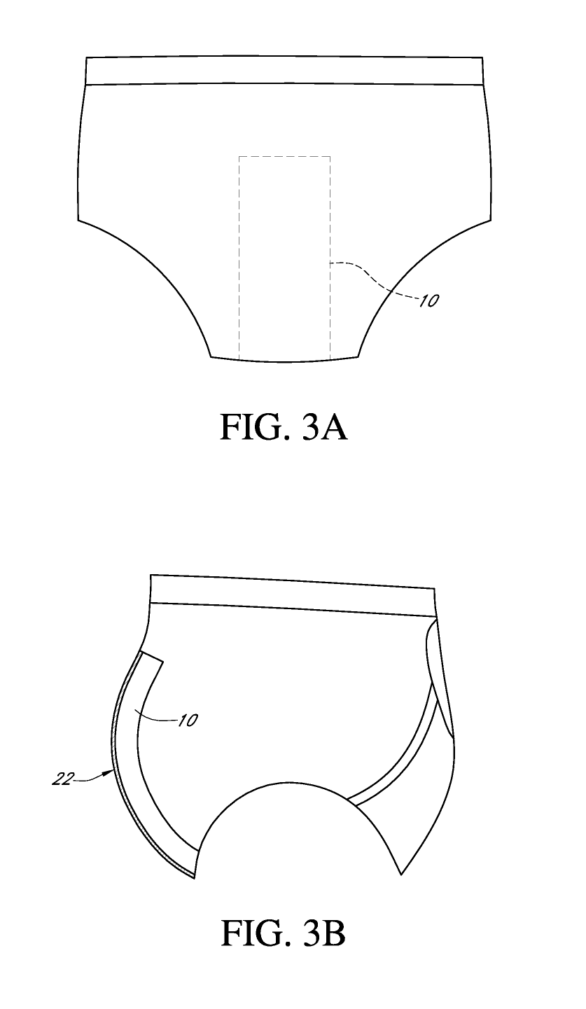

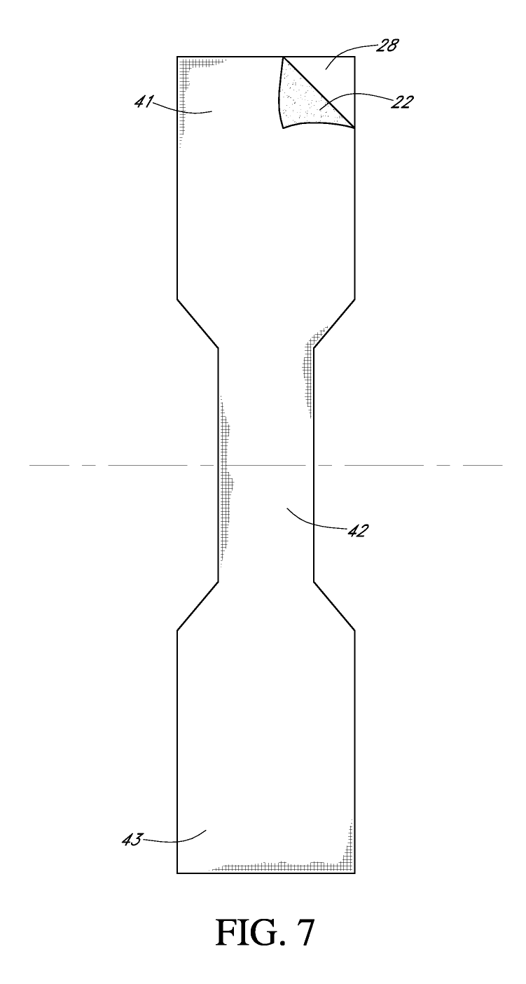

FIG. 3(A) depicts a method that the present invention is positioned on a user's undergarment. Sheet 10 is positioned in the middle and lengthwise to parallel the length of the left and right gluteus maximus crevice. FIG. 3(B) depicts a side view of the undergarment liner positioned to the rear of a user's brief so that the undergarment liner contours the user's gluteus maximus crevice. The surface of sheet 10 engages with the crevice of the user's buttocks and adhesive element 22 is attached directly with the undergarment liner. Alternatively, for those who tend to have urinary stains instead of fecal stains, the undergarment liner of the present invention may be positioned towards the user's frontal area to cover only the user's anterior area, or it may be positioned to cover both the user's anterior and posterior areas. Different variations of the present invention may be of different shapes and sizes, and may continue underneath from the posterior area to the anterior or front of the user's groin or crotch area (to protect against slight urinary discharge as well). In the embodiments of the invention depicted in FIGS. 6 and 7, sheet 10 are designed to cover both the anterior and posterior areas of the user simultaneously thereby providing dual stain prevention.

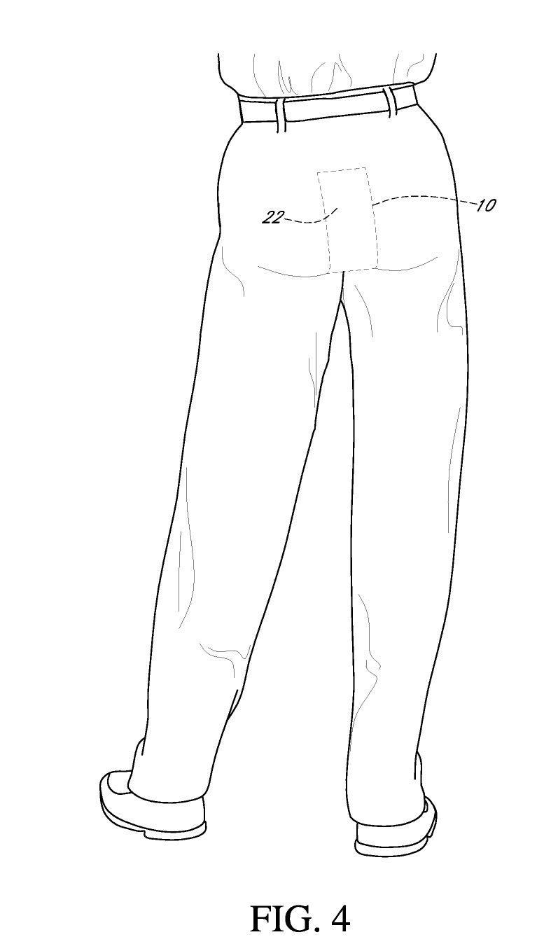

FIG. 4 depicts the application of the present invention inside of a user's pants. Such applications may be suitable for users who prefer not to wear undergarments. The positioning of sheet 10 is secured lengthwise in the middle buttocks area of the user's pants. Adhesive element 22 faces away from the user and directly comes into contact with the middle buttocks area of the pants. For user's who tend to have urinary stains instead of fecal stains, sheet 10 may be positioned towards the user's anterior area instead of user's posterior or buttocks area. Sheet 10 may be placed in any suitable position to address the user's particular staining issues.

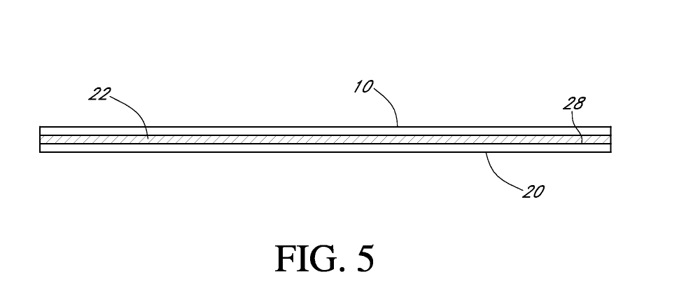

FIG. 5 depicts a magnified transverse cross-sectional view of the undergarment liner of the present invention comprising of sheet 10, adhesive element 22 is positioned between sheet 10 and peel away paper that is comprised of matte side 20 and a glossy side 28, wherein glossy side 28 of the peel away paper is removeably attached to adhesive element 22. The peel away paper covers the entire bottom side 24 of sheet 10 (see also FIG. 2). The surface of sheet 10 comes in direct contact with the user's posterior and/or anterior area when it is secured to an undergarment and worn by the user. Thus, sheet 10 is preferably made of a thin, comfortable, resilient material such as a nonwoven fabric that provides the user comfort while providing a sufficiently absorbent barrier to prevent stains from setting onto the undergarment.

To use this product, the user (or the parent of the ultimate user in embodiments of the invention adapted for use by children) must remove the peel away paper from sheet 10 and discard in order to expose adhesive element 22. Adhesive element 22 is then applied directly onto the user's undergarment or article of clothing. The present invention may be applied onto any garment, article of clothing, or undergarment that is directly exposed to a user's skin that is repeatedly exposed to fecal and/or urinary matter (e.g., briefs, boxers, panties, “pull-up” diapers or pants or shorts for those who prefer not to wear underwear). For the embodiment of the invention depicted in FIG. 1, positioning and alignment of sheet 10 usually parallels the crevice separating the buttocks to protect against fecal stains. Alternatively, sheet 10 may be positioned anterior or in front of the groin/crotch area to protect against urinary stains.

FIGS. 6 and 7 depict alternate embodiments of the present invention that address both fecal and urinary stains simultaneously wherein sheet 10 of the undergarment liner comprises: a first portion 41, a second portion 42, and a third portion 43, wherein the first portion 41 and the third portion 43 are connected by the second portion 42. Second portion 42 is positioned between first portion 41 and third portion 43 and is adapted to receive the crotch area (i.e., the area of the user adjoining the anterior and posterior regions of the user). Portion 42 is adapted to contour the inner thighs of the user since second portion 42 is narrower than both first portion 41 and third portion 43 thereby creating arches through which the inner thighs of the user pass through.

As shown in FIGS. 6 and 7, in certain embodiments of the present invention, the first portion 41 and the third portion 43 are the same shape and size thereby making the overall shape of the undergarment liner symmetrical and similar to an hour-glass shape. This hour-glass shape may be of a variety of shapes and sizes. For instance, FIG. 6 depicts a shape that is relatively wide in the rear and frontal areas (i.e., first portion 41 and third portion 43 are wide) which is suitable for larger users (or for users who produce larger stains) and provides more coverage area from stains. On the other hand, FIG. 7 illustrates an undergarment liner shape that has first portion 41 and third portion 43 that is narrower than first portion 41 and third portion 43 of the embodiment of the invention shown in FIG. 6. Variations of the invention that include narrow first portion 41 and third portion 43 are suitable for smaller users (or for users who produce smaller stains) and is more discreet from detection.

FIGS. 6 and 7 depict an embodiment adapted for placement within panty or boxer. With its hour-glass-like shape, one section of the undergarment liner may be positioned within the user's rear/posterior area to address fecal stains, and another section of the undergarment liner is positioned within the user's frontal/anterior area to address urinary stains. In one variation of the embodiments shown in FIGS. 6 and 7, first portion 41 is adapted to be placed in the posterior area of the user's underwear, second portion 42 is adapted to be placed in the crotch area of the user's underwear, and third portion 43 is adapted to be placed in the anterior area of the user's underwear. Alternatively, first portion 41 is adapted to be placed in the anterior area of the user's underwear, second portion 42 is adapted to be placed in the crotch area of the user's underwear, and third portion 43 is adapted to be placed in the posterior area of the user's underwear. First portion 41 and third portion 43 may be size, shaped and dimensioned to accommodate anterior and posterior regions of certain sexes, sizes, and body types of various users.

In other embodiments, first portion 41 and third portion 43 are of the same shape and dimension and are thus interchangeable. For example, first portion 41 is positioned within the user's rear area, and third portion 43 is positioned within the user's frontal area. Alternatively, the undergarment liner may be rotated 180 degrees since placement of the undergarment liner within the user's underwear or article of clothing is not limited to one particular position. As such, third portion 43 may be positioned within the user's rear area and first portion 41 may be positioned within the user's frontal area.

As in the embodiment depicted in FIGS. 1-5, sheet 10 engages with the user and is the surface that receives any fecal or urinary stains, and glossy side 28 of the peel away paper is initially attached to adhesive element 22 until the peel away paper is peeled away and adhesive element 22 of sheet 10 is applied directly to the user's undergarment or article of clothing.

In alternate embodiments, wherein sheet 10 is not symmetrical, first portion 41 may be larger and/or of a different shape than third portion 43 or first portion 41 may be smaller and/or of a different shape than third portion 43. In some variations of the invention, first portion 41 may be specifically adapted for use with the rear or posterior area of the user, and third portion 43 may be specifically adapted for use with the front or anterior area of the user. Alternatively, first portion 41 may be specifically adapted for use with the front or anterior area of the user, and third portion 43 may be specifically adapted for use with the rear or posterior area of the user

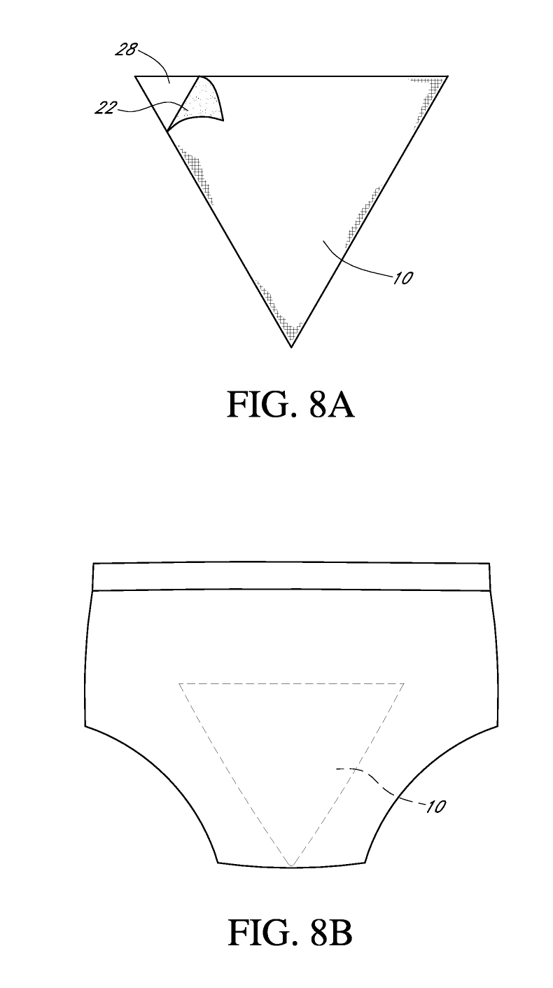

FIGS. 8(a) and 8(b) depict another embodiment of the present invention wherein the shape of the undergarment liner is triangular. Referring to FIG. 8(a), sheet 10 is triangular in shape and engages with the user as it is the surface that receives any fecal or urinary stains, and glossy side 28 of the peel away paper is initially in contact with adhesive element 22 until it is peeled away and sheet 10 is secured to the undergarment by applying adhesive element 22 onto to the user's undergarment or article of clothing. Referring to FIG. 8(b), in such triangular-shaped embodiments, sheet 10 of the undergarment liner may be placed more towards the rear area of the underwear or article of clothing (to prevent fecal stains) or more towards the frontal area of the underwear or article of clothing (to prevent urinary stains). Accordingly, FIG. 8(b) may depict either a rear view or a front view of an undergarment with sheet 10 shown in FIG. 8(a) therein and illustrates how sheet 10 of the undergarment liner is positioned onto the user's underwear depending on whether fecal or urinary stains are to be addressed. The triangular-shaped embodiments provide a versatile alternative allowing either fecal or urinary stains to be addressed since some users do not have both fecal and urinary stain issues. If the user does have both fecal and urinary stain issues, then the hour-glass-shaped embodiments illustrated in FIGS. 6 and 7 would be able to simultaneously address such issues.

### Further Embodiments

Although the present invention has been described above with reference to certain versions thereof as depicted in the drawings, other versions are possible. For instance, in addition to being specifically adapted for use by either men or by children, certain embodiments of the invention may be adapted for use by women. Unlike typical panty liners currently on the market, the undergarment liner of the present invention has a larger surface area making it possible for women to also use the liners to address fecal stains or both fecal and urinary stains. The thin, sleek, and discreet features of the liners are ideal for women who have issues with stains but desire to wear thin, tight, or revealing clothes through which normal panty liners would easily be detected.

Variations of the present invention may be of different sizes (appropriate for different sized men, children of different ages, children of different sizes and weights, fitted for different kinds of boys' or girls' undergarments, different thicknesses and absorbencies, different materials, made with different scents, with different types of adhesive backing (perhaps based on the material of the undergarment it is to be applied to or based on the activity of the user), and even adorned with different coloring, logos, or cartoon characters or superhero designs to appeal to children.

In alternate embodiments of the invention, variations can comprise of biodegradable, flushable material, organic material, recycled paper/paper products, paper products, any woven cloth material, or cotton or other similar textile. Sizes can vary, and be greater or less than 8 cm wide×28 cm in length, in order to accommodate for extra width and length to prevent stains from mild urinary discharge, or very small to accommodate children's sizes. Shapes can range anywhere from straight-edged rectangle, flattened oval, disk-shaped, semi-circular, parabola-shaped, T-shaped, H-shaped, U-shaped, barbell-shaped, soft curved edges, panty outlined cut-outs or brief style outlined cut-outs to specifically match the shape of the appropriate undergarment, or any other appropriate shape to protect the undergarment from stains while providing for the user's comfort. Adhesive variations can include polyurethane, polyvinyl acetate (PVA's), pressure sensitive adhesives, water based adhesives, vegetable glues, silicone adhesives, film adhesives, or variations thereof. Child-friendly adhesives may be used, and variations of this product may be designed to be even more simple to attach (for example, with directions and diagrams on the peel away paper itself) in order to promote attachment by children themselves. This invention can be specifically designed for children of all ages and both sexes. The present invention may further include additional structures and features including aesthetically pleasing features such as various designs and colors, various logos, cartoon characters, or superhero figures to appeal to children. It may also be customized or adapted for use by certain user body-types and sizes, and may also include deodorant, antiperspirant, fragrance, activated charcoal, talcum powder, baking soda, or other fragrance-producing or odor-absorbing materials.

While the description above refers to particular embodiments of the present invention, it will be understood that many modifications may be made without departing from the spirit thereof. The presently disclosed embodiments are therefore to be considered in all respects as illustrative and not restrictive.

What is claimed is:

1. A disposable undergarment liner for preventing undergarment stains, the undergarment liner comprising:
a nonwoven sheet comprised of
a top side,
a bottom side, and
an adhesive element permanently affixed to the bottom side; and
a peel away paper that covers the adhesive element wherein the peel away paper is comprised of
a glossy side removeably attached to the adhesive element and
a matte side that faces opposite the top side wherein the peel away paper is removed to expose the adhesive element and the adhesive element adheres directly onto a user's undergarment.

2. The undergarment liner of claim 1 wherein the nonwoven sheet is comprised of a rectangular shape.

3. The undergarment liner of claim 1 wherein the nonwoven sheet is comprised of a triangular shape.

4. The undergarment liner of claim 1 wherein the nonwoven sheet further comprises a first portion, a second portion, and a third portion wherein the second portion is positioned between the first portion and the third portion and the second portion is narrower than the first portion and the third portion.

5. The undergarment liner of claim 4 wherein the first portion is adapted to be placed in the posterior area of the user's undergarment, the second portion is adapted to be placed in the crotch area of the user's undergarment, and the third portion is adapted to be placed in the anterior area of the user's undergarment.

6. The undergarment liner of claim 1 wherein the nonwoven sheet further comprises a second layer positioned below the top side and above the bottom side wherein the second layer is comprised of a material that is more dense than the nonwoven sheet.

7. The undergarment liner of claim 6 wherein the material is comprised of a thin plastic sheet.

8. A men's disposable undergarment liner for preventing undergarment stains, the undergarment liner comprising:
a nonwoven sheet comprised of
a top side,
a bottom side, and
an adhesive element permanently affixed to the bottom side; and
a peel away paper that covers the adhesive element wherein the peel away paper is comprised of
a glossy side removeably attached to the adhesive element and
a matte side that faces opposite the top side
wherein the peel away paper is removed to expose the adhesive element and the adhesive element adheres directly onto a user's undergarment.

9. The men's disposable undergarment liner of claim 8 wherein the nonwoven sheet is comprised of a rectangular shape.

10. The men's disposable undergarment liner of claim 8 wherein the nonwoven sheet further comprises a first portion, a second portion, and a third portion wherein the second portion is positioned between the first portion and the third portion and the second portion is narrower than the first portion and the third portion.

11. The men's disposable undergarment liner of claim 8 wherein the nonwoven sheet further comprises a second layer positioned below the top side and above the bottom side wherein the second layer is comprised of a material that is more dense than the nonwoven sheet.

12. The men's disposable undergarment liner of claim 11 wherein the material is comprised of a thin plastic sheet.

13. A children's disposable undergarment liner for preventing undergarment stains, the undergarment liner comprising:
a nonwoven sheet comprised of
a top side,
a bottom side, and
an adhesive element permanently affixed to the bottom side; and
a peel away paper that covers the adhesive element wherein the peel away paper is comprised of
a glossy side removeably attached to the adhesive element and
a matte side that faces opposite the top side wherein the peel away paper is removed to expose the adhesive element and the adhesive element adheres directly onto a user's undergarment.

14. The children's disposable undergarment liner of claim 13 wherein the nonwoven sheet is comprised of a rectangular shape.

15. The children's disposable undergarment liner of claim 13 wherein the nonwoven sheet is comprised of a triangular shape.

16. The children's disposable undergarment liner of claim 13 wherein the nonwoven sheet further comprises a first portion, a second portion, and a third portion wherein the second portion is positioned between the first portion and the third portion and the second portion is narrower than the first portion and the third portion.

17. The children's disposable undergarment liner of claim 16 wherein the first portion and the third portion are the same shape and dimension.

18. The children's disposable undergarment liner of claim 16 wherein the first portion is adapted to be placed in the posterior area of the user's undergarment, the second portion is adapted to be placed in the crotch area of the user's undergarment, and the third portion is adapted to be placed in the anterior area of the user's undergarment.

19. The children's disposable undergarment liner of claim 13 wherein the nonwoven sheet further comprises a second layer positioned below the top side and above the bottom side wherein the second layer is comprised of a material that is more dense than the nonwoven sheet.

20. The children's disposable undergarment liner of claim 13 wherein the material is comprised of a thin plastic sheet.
